(19) United States
(12) Patent Application Publication
(10) Pub. No.: US 2019/0038008 A1
(43) Pub. Date: Feb. 7, 2019

(54) STORAGE BOTTLE WITH FILTRATION AND DISINFECTION EFFECTS

(71) Applicant: An-Jen LEE, Miaoli County (TW)

(72) Inventor: An-Jen LEE, Miaoli County (TW)

(21) Appl. No.: 15/666,634

(22) Filed: Aug. 2, 2017

(51) Int. Cl. A45F 3/18; B65D 47/06; B65D 51/28; B65D 41/04; B65D 85/72; A61L 2/10

(52) U.S. Cl. CPC A45F 3/18; B65D 47/06; B65D 51/28; A45F 2003/163; B65D 85/72; A61L 2/10; B65D 41/04

(57) ABSTRACT

A storage bottle with filtration and disinfection effects has an outer casing, a cap, a bottom base, and a waterproof board. The outer casing is hollow. The cap is detachably connected to the outer casing and has an assembling recess and a filter group. The assembling recess communicates with the outer casing. The filter group is deposited in the assembling recess and has at least one filtering layer. The bottom base is detachably connected to the outer casing and has a mounting recess and a sterilization group. The sterilization group is deposited in the mounting recess and has at least one light emitting diode. The at least one light emitting diode emits an ultraviolet light with a sterilization band to liquid in the outer casing. The waterproof board is deposited between the outer casing and the bottom base to close the mounting recess of the bottom base.

### BACKGROUND OF THE INVENTION

### 1. Field of the Invention

The present invention relates to a storage bottle, and more particularly to a storage bottle that may be carried easily, may be used conveniently, and may provide filtering and sterilization effects.

### 2. Description of Related Art

70% of the human body is composited by water, so most of the organs and tissues need to rely on water, which shows the importance of water for the mankind. Additionally, with the development and advance of science and technology, requirements on drinking water are also raised to higher standards. A conventional water purifier or water purification equipment is mainly composited by water treatment equipment, disinfection equipment and other devices to provide people with the necessary drinking water.

However, the conventional water purifier or water purification equipment has a certain volume, and most of the conventional water purifier or water purification equipment is fixed at a specific location for people to use. Therefore, users need to move to the location to store or drink water, and this is inconvenient in use. Moreover, when people participate in activities or move outdoors, although people can carry bottled water to achieve the purpose of drinking water, but the carried bottled water will not only increase the weight and can only store a limited amount of water, and the bottles after use will also have an impact on the environment.

To overcome the shortcomings, the present invention provides a storage bottle with filtration and disinfection effect to mitigate or obviate the aforementioned problems.

### SUMMARY OF THE INVENTION

The primary objective of the present invention is to provide a storage bottle that that may be carried easily, may be used conveniently, and may provide filtering and sterilization effects.

The storage bottle with filtration and disinfection effect in accordance with the present invention has an outer casing, a cap, a bottom base, and a waterproof board. The outer casing is hollow. The cap is detachably connected to the outer casing and has an assembling recess and a filter group. The assembling recess communicates with the outer casing. The filter group is deposited in the assembling recess and has at least one filtering layer. The bottom base is detachably connected to the outer casing and has a mounting recess and a sterilization group. The sterilization group is deposited in the mounting recess and has at least one light emitting diode. The at least one light emitting diode emits an ultraviolet light with a sterilization band to liquid in the outer casing. The waterproof board is deposited between the outer casing and the bottom base to close the mounting recess of the bottom base.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a storage bottle in accordance with the present invention;

FIG. 2 is a partial perspective view of the storage bottle in FIG. 1, shown with some internal components;

FIG. 3 is a perspective view in partial section of the storage bottle in FIG. 1;

FIG. 4 is an exploded perspective view of the storage bottle in FIG. 1;

FIG. 5 is a side view in partial section of the storage bottle in FIG. 1;

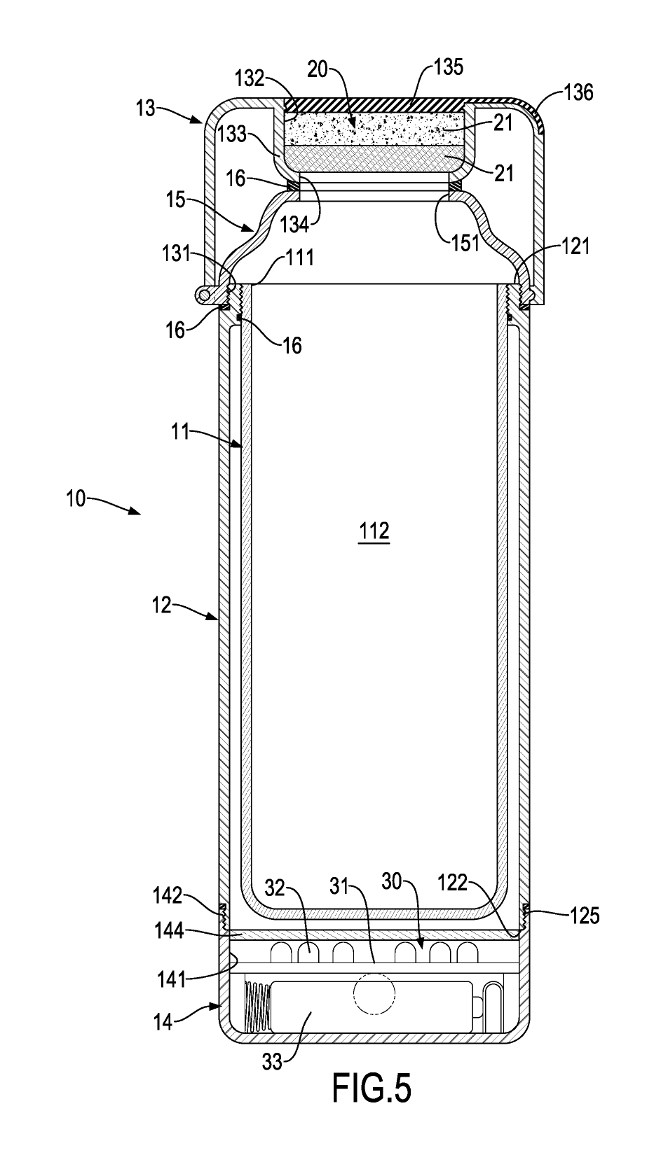

FIG. 6 is an operational perspective view of the storage bottle in FIG. 1;

FIG. 7 is an operational side view in partial section of the storage bottle in FIG. 1;

FIG. 8 is a perspective view of a second embodiment of a storage bottle in accordance with the present invention;

FIG. 9 is a side view in partial section of the storage bottle in FIG. 8; and

FIG. 10 is an operational perspective view of the storage bottle in FIG. 8.

### DETAILED DESCRIPTION OF THE PREFERRED EMBODIMENT

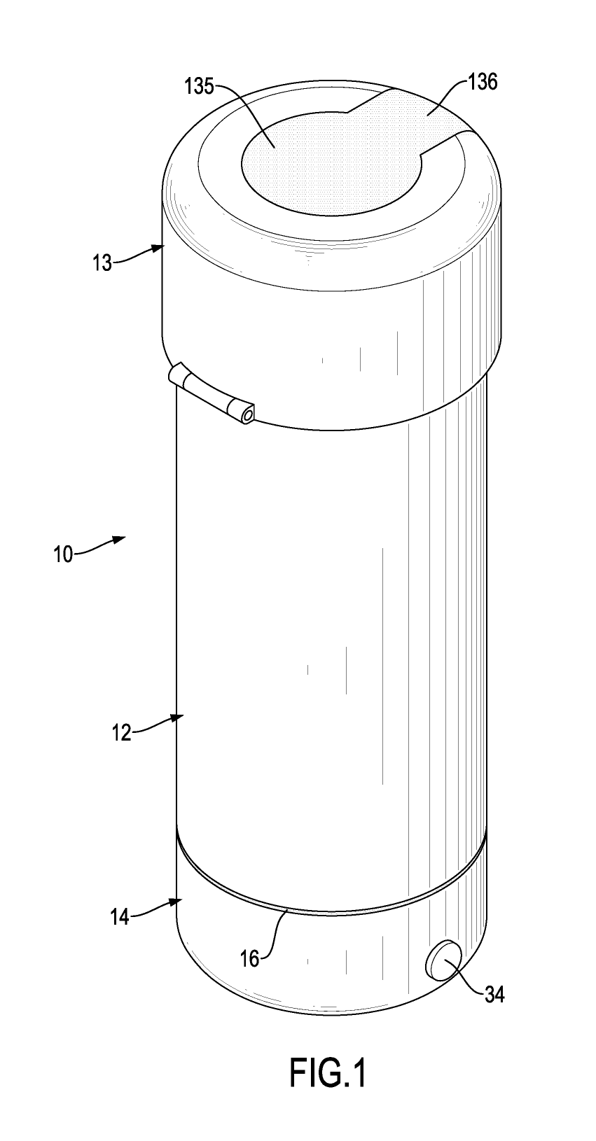

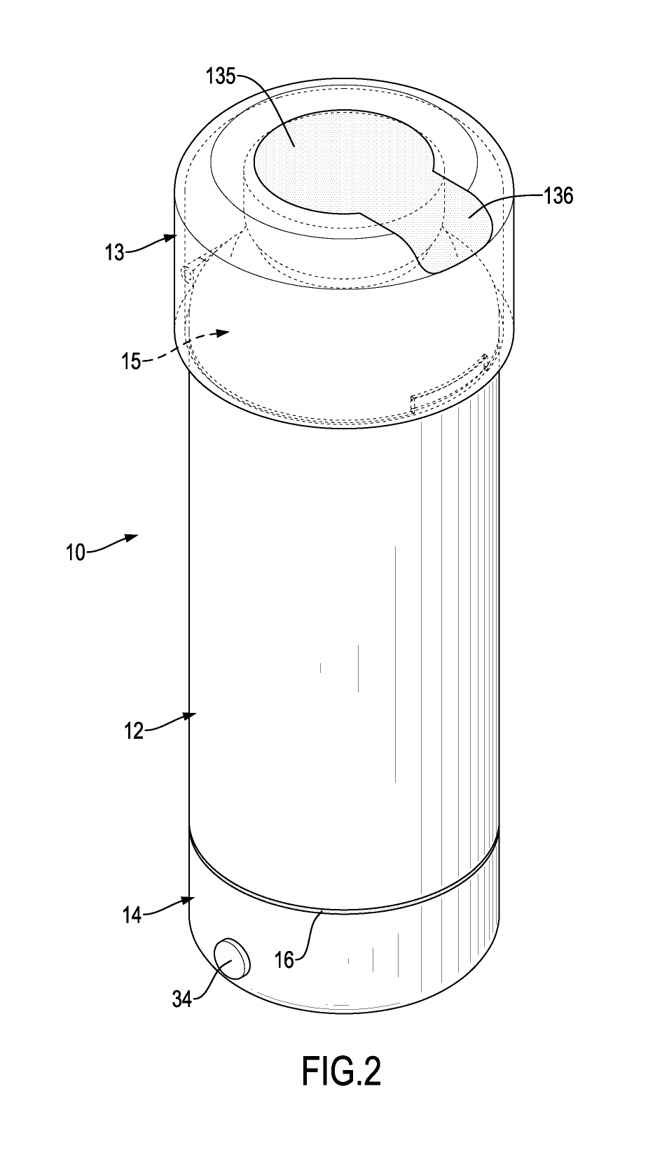

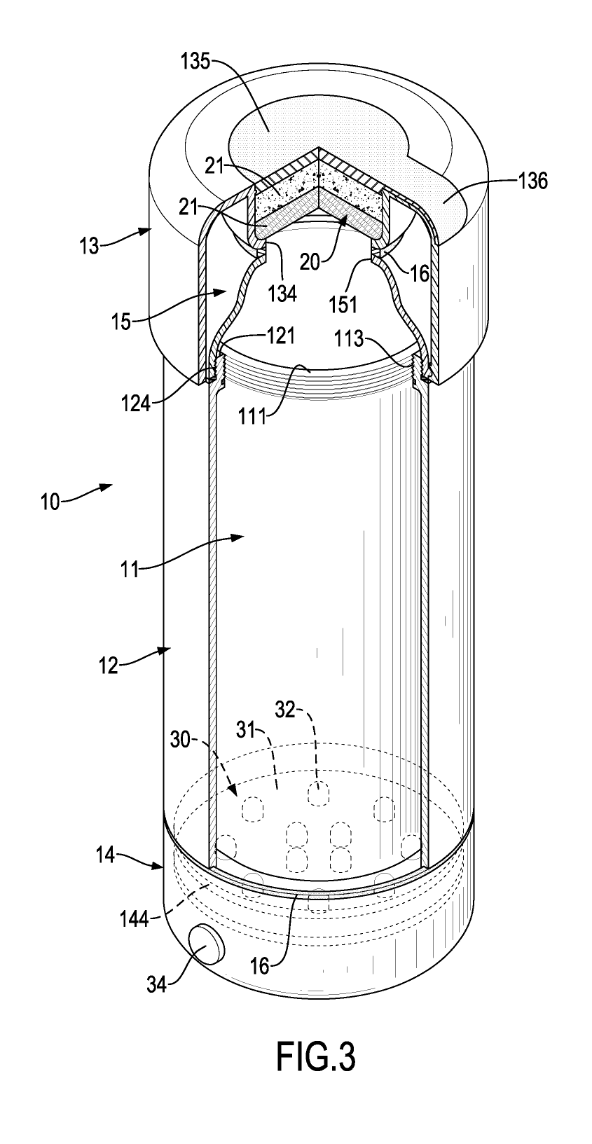

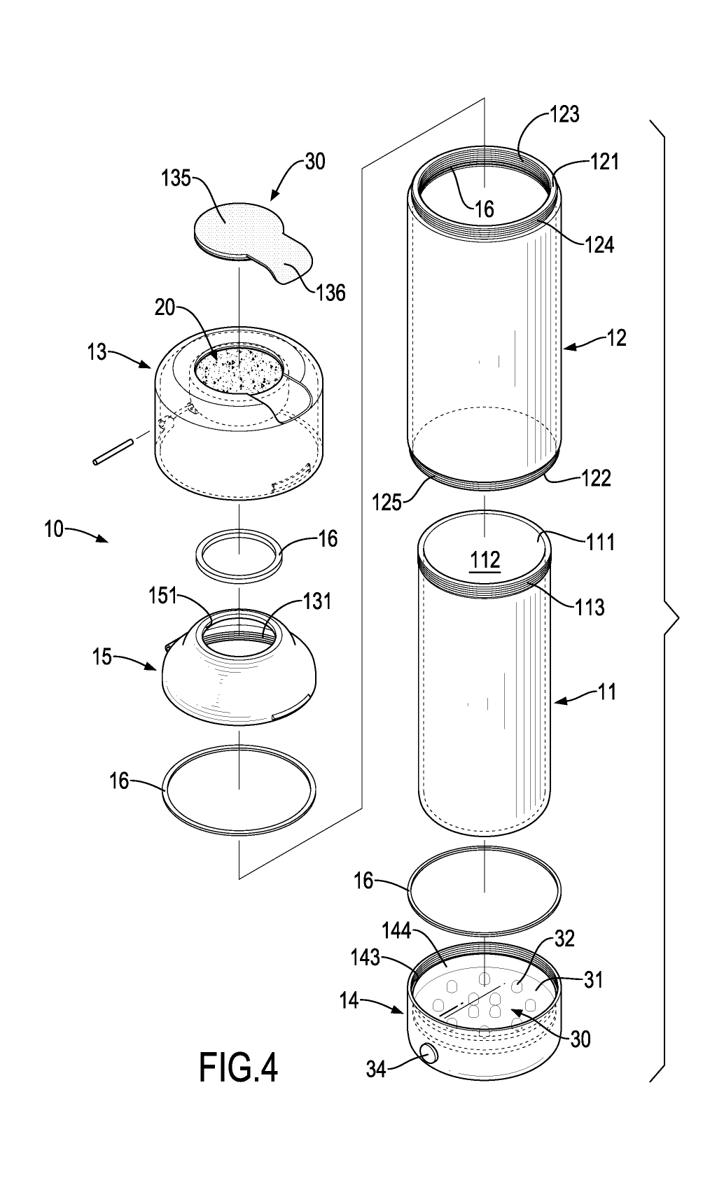

With reference to FIGS. 1 to 4, a first embodiment of a storage bottle with filtration and disinfection effects in accordance with the present invention has a bottle group 10, a filter group 20, and a sterilization group 30.

The bottle group 10 has an inner body 11, an outer casing 12, a cap 13, and a bottom base 14. The inner body 11 is hollow, and has a top end, a bottom end, an external surface, an opening 111, a chamber 112, and an engaging structure 113. The bottom end of the inner body 11 is closed. The opening 111 is formed through the top end of the inner body 11. The chamber 112 is deposited in the inner body 11 between the top end and the bottom end of the inner body 11, and communicates with the opening 111. The engaging structure 113 is deposited around the external surface of the inner body 11 adjacent to the opening 111.

The outer casing 12 is hollow, is mounted around and connected to the inner body 11, and has a top end, a bottom end, an upper flange 121, a lower flange 122, an inner engaging segment 123, an outer engaging segment 124, and an engaging element 125. The upper flange 121 is axially and annularly formed on and protrudes from the top end of the outer casing 12, and has an inner surface and an outer surface. The lower flange 122 is axially and annularly formed on and protrudes from the bottom end of the outer casing 12, is deposited below the bottom end of the inner body 11, and has an outer surface. The inner engaging segment 123 is deposited on the inner surface of the upper flange 121, and selectively engages with the engaging structure 113 of the inner body 11 to hold the inner body 11 in the outer casing 12. The outer engaging segment 124 is deposited on the outer surface of the upper flange 121. The engaging element 125 is deposited on the outer surface of the lower flange 122.

The cap 13 is detachably connected to the outer casing 12 above the inner body 11 to close the opening 111 of the inner body 11 and to prevent liquid in the chamber 112 from flowing out of the bottle group 10, and has a bottom end, a middle, a top end, a locking portion 131, an assembling recess 132, an assembling segment 133, and a through hole 134. The locking portion 131 is deposited on the bottom end of the cap 13, and selectively engages with the outer engaging segment 124 on the upper flange 121 of the outer casing 12. The assembling recess 132 is deposited in the cap 13 for storage of the filter group 20, and communicates with the chamber 112 of the inner body 11 via the opening 111 of the inner body 11. The assembling segment 133 is deposited on the middle of the cap 13, the through hole 134 is formed through a bottom of the assembling segment 133, and the assembling recess 132 is deposited in the assembling segment 133 and communicates with the chamber 112 of the inner body 11 via the through hole 134 and the opening 111 of the inner body 11.

Furthermore, in the first embodiment of the storage bottle in accordance with the present invention, the assembling segment 133 is an annular protruding wall, and is formed on and protrudes downwardly from the top end of the cap 13. The assembling recess 132 is deposited in the annular protruding wall, and the through hole 134 is formed through a bottom of the annular protruding wall. In addition, the cap 13 has a covering slice 135 deposited on the top end of the cap 13 to cover the annular protruding wall. The covering slice 135 has a free end and a flip-up portion 136. The free end of the covering slice 135 extends radially away from the annular protruding wall, and the flip-up portion 136 is deposited on the free end of the covering slice 135.

In addition, in the first embodiment of the storage bottle in accordance with the present invention, the cap 13 further has a bottle mouth 15 pivotally connected to the cap 13 and connected to the outer casing 12, and the locking portion 131 of the cap 13 is deposited on an inner surface of the bottle mouth 15 adjacent to a bottom of the bottle mouth 15. Then, the bottle mouth 15 is deposited on the outer casing 12 above the inner body 11 by the locking portion 131 connecting with the outer engaging segment 124 of the outer casing 12. The bottle mouth 15 has a water hole 151 formed through a top end of the bottle mouth 15 below the through hole 134 of the cap 13, and this enables the assembling recess 132 of the cap 13 to communicate with the chamber 112 of the inner body 11 via the through hole 134, the water hole 151, and the opening 111 of the inner body 11.

The bottom base 14 is detachably connected to the outer casing 12 below the inner body 11, and has a top end, a bottom end, a mounting recess 141, a protruding edge 142, an engaging portion 143, and a waterproof board 144. The mounting recess 141 is deposited in the bottom base 14 between the top end and the bottom end. of the bottom base 14. The protruding edge 142 is axially and annularly formed on and protrudes from the top end of the bottom base 14, and is connected to the lower flange 122 of the outer casing 12 to hold the bottom base 14 below the inner body 11 at a spaced interval. The engaging portion 143 is deposited on an inner surface of the protruding edge 142 and is selectively connected to the engaging element 125 of the lower flange 122. Furthermore, the structural configurations of the engaging structure 113, the inner engaging segment 123, the outer engaging segment 124, the engaging element 125, the locking portion 131, and the engaging portion 143 are not limited in the present invention, and the relative structures that can be combined with each other also belong to the structural configurations defined in the present invention, such as inner threads, outer threads, protrusions, recesses, etc.

The waterproof board 144 is transparent, and is transversally deposited in the bottom base 14 below the protruding edge 142 to close the mounting recess 141. Additionally, the bottle group 10 has at least one sealing ring 16 deposited in each one of the components between the inner body 11 and the outer casing 12, between the outer casing 12 and the cap 13, between the outer casing 12 and the bottom base 14, and between the bottle mouth 15 and the assembling segment 133, and this may prevent the liquid in the bottle group 10 from flowing out of the storage bottle.

The filter group 20 is connected to the bottle group 10 and has at least one filtering layer 21. The at least one filtering layer 21 is deposited in the assembling recess 132 of the cap 13. When water flows into the inner body 11 via the cap 13, the water is filtered by the at least one filtering layer 21 and flows in the chamber 112 via the through hole 134, the water hole 151, and the opening 111 of the inner body 11, and this may provide a filtering effect to the water. Furthermore, in the first embodiment of the present invention, the filter group 20 has two filtering layers 21 stacked with each other and deposited in the assembling recess 132, and the two filtering layers 21 are made of different filter materials to filter different contaminants such as suspension or particles. In addition, each filtering layer 21 is an activated carbon layer, a cotton filter layer, a fiber filter layer, a ceramic layer or a silver layer.

The sterilization group 30 is connected to the bottle group 10 and has a circuit board 31, at least one light emitting diode (LED) 32, and a power supply 33. The circuit board 31 is deposited in the mounting recess 141 of the bottom base 14 below the protruding edge 142 before the bottom base 14 is connected to the outer casing 12. The at least one LED 32 is electrically deposited on a top side of the circuit board 31 toward the bottom end of the inner body 11, and emits a light source to the inner body 11. The power supply 33 is deposited on a bottom side of the circuit board 31 and is electrically connected to the circuit board 31 to provide electricity to the at least one LED 32.

Additionally, the sterilization group 30 has multiple LEDs 32 deposited on the top side of the circuit board 31 at spaced intervals, and each one of the multiple LEDs 32 emits an ultraviolet light with a sterilization band to the bottom end of the inner body 11. Further, the sterilization group 30 has a switch 34 deposited on an external surface of the bottom base 14 and electrically connected to the power supply 33 and the circuit board 31.

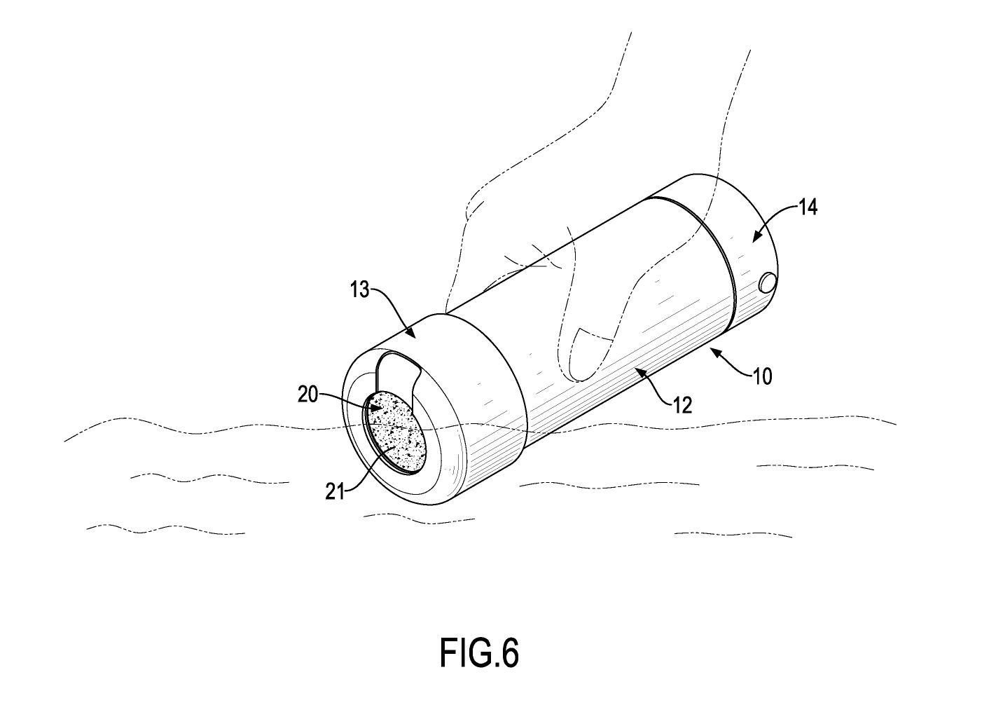

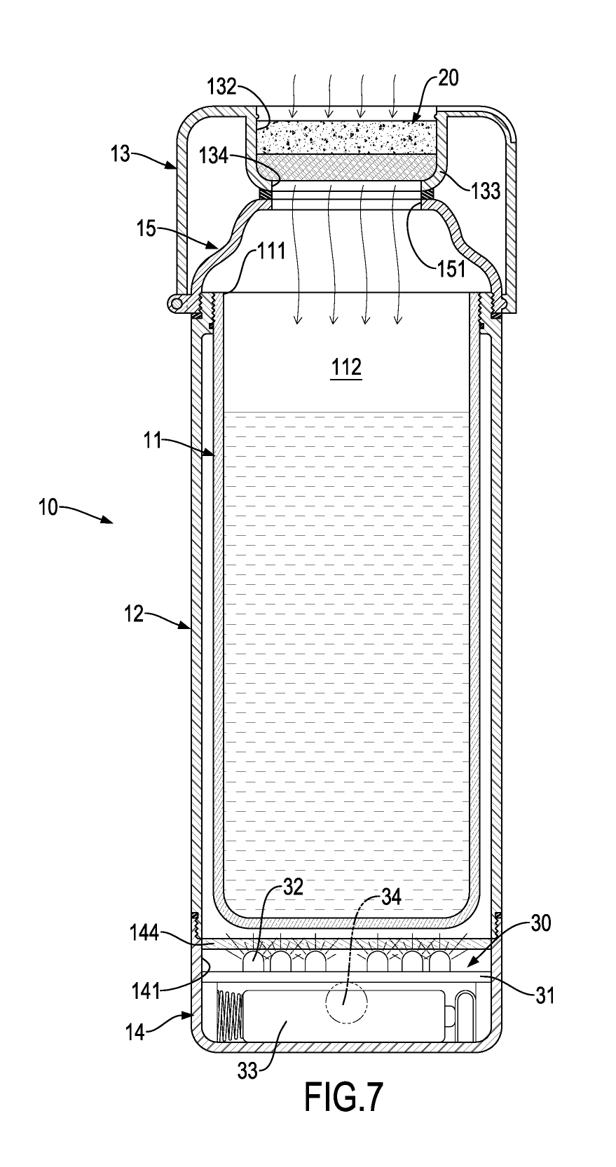

With reference to FIGS. 1, 6, and 7, when the storage bottle of the first embodiment in accordance with the present invention is in use, the covering slice 135 is separated from the cap 13 by pulling the flip-up portion 136 to expose the filter group 20 out of the top end of the cap 13. Then, a user may deposit the bottle group 10 obliquely in the stream or below the liquid to enable water or liquid to be filtered by each filtering layer 21 of the filter group 20 at the cap 13. After the above-mentioned filtering process, the water or liquid flows in and is stored in the chamber 112 of the inner body 11 via the through hole 134, the water hole 151, and the opening 111 of the inner body 11. At this time, the switch 34 is pressed to start the sterilization group 30, and each LED emits an ultraviolet light with a sterilization band to the inner body 11 and to disinfect and sterilize the water or liquid in the inner body 11.

In addition, the user may separate the inner body 11 from the outer casing 12, and may store the water that is filtered by the filter group 20 directly in the outer casing 12, and may disinfect and sterilize the water by the sterilization group 30. After the completion of filtration and disinfection, the user may pull and rotate the cap 13 relative to the bottle mouth 15, and drink the water or liquid via the water hole 151 after filtering by the filter group 20 and sterilizing by the sterilization group 30, and this may provide a filtering effect (from outside to inside) and an internal sterilizing effect to the water or liquid and is convenient in use, and the storage bottle can be carried by the user easily. Furthermore, when the inner body 11 is deposited in the outer casing 12, an insulating effect can be provided to the water or liquid that is stored in the inner body 11. Therefore, the inner body 11 can be either adopted or omitted according to the needs of the user.

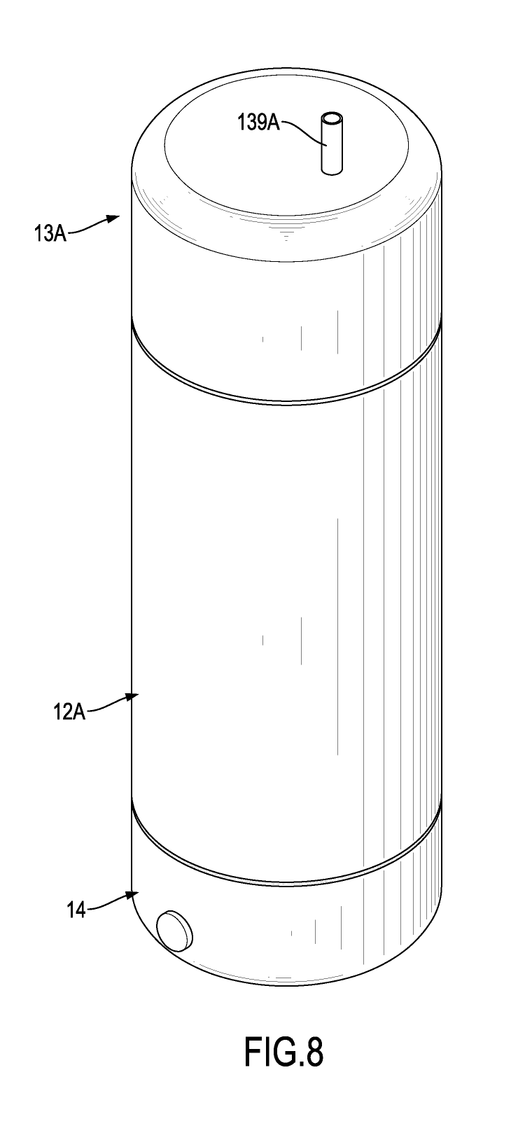

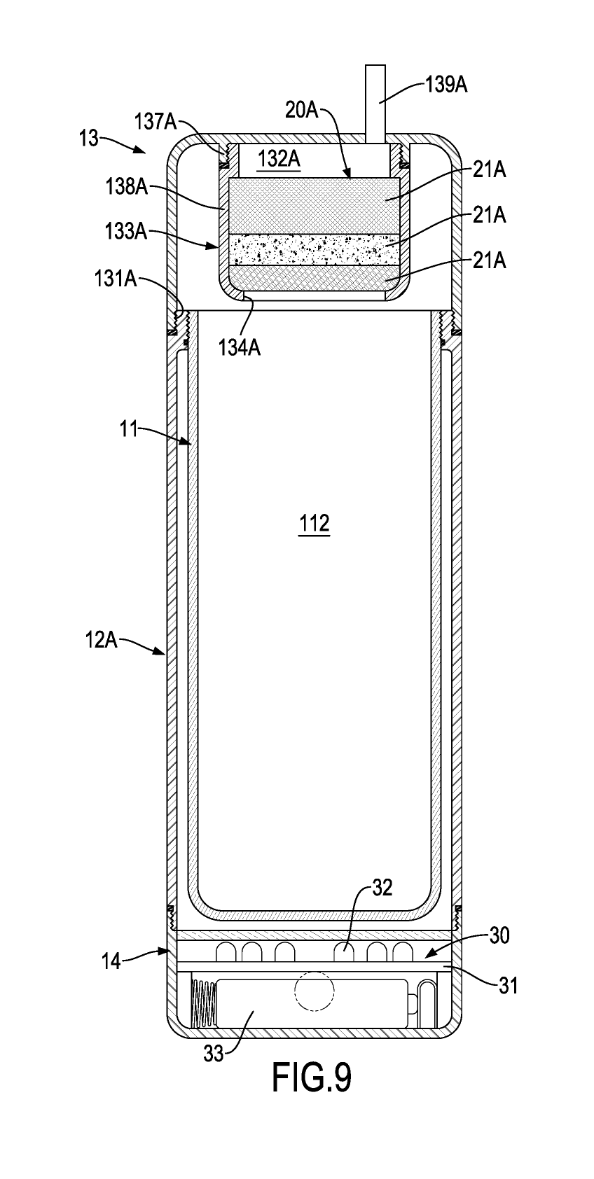

With reference to FIGS. 8 and 9, a second embodiment of a storage bottle in accordance with the present invention is substantially the same as the first embodiment except for the following features. The cap 13A of the second embodiment does not have the bottle mouth 15 of the first embodiment, and the cap 13A is directly connected to the outer casing 12A. The assembling segment 133A of the cap 13A has a connecting flange 137A and an assembling convex wall 138A. The connecting flange 137A is axially and annularly formed on and protrudes downwardly from an inner side of the top end of the cap 13A. The assembling convex wall 138A is detachably connected to the connecting flange 137A. The assembling recess 132A is deposited between the connecting flange 137A and the assembling convex wall 138A, and the through hole 134A is formed through a bottom of the assembling convex wall 138A. Further, the cap 13A has a straw 139A deposited on the top end of the cap 13A and communicating with the assembling recess 132A, and the filter group 20A has three filtering layers 21A stacked with each other and deposited in the assembling recess 132A of the cap 13A.

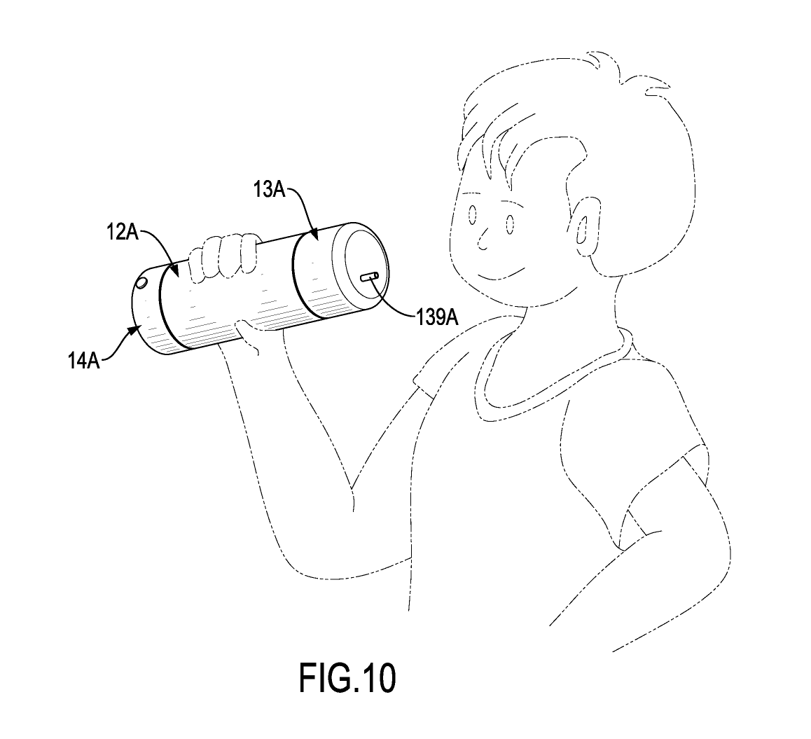

With reference to FIGS. 8, 9, and 10, when the storage bottle of the second embodiment of the present invention is in use, the cap 13A is rotated relative to the outer casing 12A and to separate from the outer casing 12A. Then, the opening 111 of the inner body 11 communicates with the external environment, water or liquid without filtering and sterilizing flows and is stored in the chamber 112 of the inner body 11 via the opening 111. After sufficient water or liquid is stored in the inner body 11, the cap 13A is connected to the outer casing 12A again, and the sterilization group 30 is started to enable each LED 32 to emit an ultraviolet light to disinfect and sterilize the water or liquid in the inner body 11. The user may tilt the bottle group 10 after the completion of filtration and disinfection, and the water or liquid in the chamber 112 may flow to the straw 139A via the through hole 134A of the assembling convex wall 138A of the assembling segment 133A and each filtering layer 21A. Then, the user may drink the water or liquid via the straw 139A. after filtering by the filter group 20 and sterilizing by the sterilization group 30, and this may provide a filtering effect (from inside to outside) and an internal sterilizing effect to the water or liquid, and is convenient in use and can be carried by the user easily.

According to the above-mentioned features and structural relationships of the storage bottle, the filter group 20 is deposited in the cap 13, 13A to provide a filtering effect (from outside to inside or from inside to outside) to the water or liquid that flows in or out of the bottle group 10. Furthermore, the sterilization. group 30 may provide a sterilizing effect to the water or liquid that is stored in the inner body 11 or the outer casing 12, 12A. Then, the user may drink the clean water or liquid after filtering, disinfecting, and sterilizing. In addition, the structure of the storage bottle is simple, and the storage bottle can be carried easily and conveniently. The user may drink water or liquid after filtering, disinfecting, and sterilizing according to the user's need without being limited to specific locations.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A storage bottle with filtration and disinfection effects having:
an outer casing being hollow;
a cap detachably connected to the outer casing above the outer casing, and having
an assembling recess deposited in the cap and communicating with the outer casing; and
a filter group deposited in the assembling recess and having at least one filtering layer;
a bottom base detachably connected to the outer casing below the outer casing, and having
a mounting recess deposited in the bottom base; and
a sterilization group deposited in the mounting recess and having at least one light emitting diode emitting an ultraviolet light with a sterilization band to liquid in the outer casing; and
a waterproof board deposited between the outer casing and the bottom base to close the mounting recess of the bottom base.

2. The storage bottle as claimed in claim 1, wherein the waterproof board is deposited in the bottom base to dispose the sterilization group between the bottom base and the waterproof board.

3. The storage bottle as claimed in claim 2, wherein
the storage bottle has an inner body mounted in the outer casing;
the inner body has
a top end;
a bottom end;
an opening formed through the top end of the inner body; and
a chamber deposited in the inner body between the top end and the bottom end of the inner body, and communicating with the opening;
the assembling recess of the cap communicates with the chamber via the opening of the inner body;
the liquid is stored in the chamber of the inner body after filtering by the at least one filtering layer; and
the ultraviolet light with a sterilization band of the sterilization group is emitted to the inner body via the waterproof board.

4. The storage bottle as claimed in claim 3, wherein
the cap has an assembling segment deposited in a middle of the cap;
the assembling segment has a through hole formed through a bottom of the assembling segment; and
the assembling recess is deposited in the assembling segment, and communicates with the chamber of the inner body via the through hole and the opening of the inner body.

5. The storage bottle as claimed in claim 4, wherein
the assembling segment is an annular protruding wall, and is formed on and protrudes downwardly from a top end of the cap;
the assembling recess is deposited in the annular protruding wall; and
the through hole is formed through the bottom of the assembling segment.

6. The storage bottle as claimed in claim 5, wherein
the cap has a bottle mouth pivotally connected to the cap and connected to the outer casing;
the bottle mouth has a water hole formed through a top end of the bottle mouth;
the assembling recess of the cap communicates with the chamber of the inner body via the through hole, the water hole, and the opening of the inner body; and
the liquid flows in the inner body via the cap, is filtered by the at least one filtering layer, and is stored. in the chamber via the through hole, the water hole, and the opening of the cap.

7. The storage bottle as claimed in claim 6, wherein
the cap has a covering slice deposited on the top end of the cap to cover the annular protruding wall; and
the covering slice has
a free end extending radially away from the annular protruding wall; and
a flip-up portion deposited on the free end of the covering slice.

8. The storage bottle as claimed in claim 7, wherein
the sterilization group has
a power supply;
a circuit board mounted in the mounting recess of the bottom base and electrically connected to the power supply; and
a switch deposited on the bottom base and electrically connected to the power supply; and
the sterilization group has multiple light emitting diodes deposited on a top side of the circuit board at spaced intervals.

9. The storage bottle as claimed in claim 4, wherein
the assembling segment of the cap has
a connecting flange axially and annularly formed on and protruding downwardly from an inner side of a top end of the cap; and
an assembling convex wall detachably connected to the connecting flange;
the assembling recess is deposited between the connecting flange and the assembling convex; and
the through hole is formed through a bottom of the assembling convex wall.

10. The storage bottle as claimed in claim 3, wherein
the inner body has an engaging structure deposited around an external surface of the inner body adjacent to the opening;
the outer casing has
a top end;
a bottom end;
an upper flange axially and annularly formed on and protruding from the top end of the outer casing, and having an inner surface and an outer surface;
a lower flange axially and annularly formed on and protruding from the bottom end of the outer casing, deposited below the inner body, and having an outer surface;
an inner engaging segment deposited on the inner surface of the upper flange, and selectively engaging with the engaging structure of the inner body to hold the inner body in the outer casing;
an outer engaging segment deposited on the outer surface of the upper flange; and
an engaging element deposited on the outer surface of the lower flange;
the bottom base has
a protruding edge axially and annularly formed on and protruding from a top end of the bottom base, and connected to the lower flange of the outer casing to hold the bottom base below the inner body at a spaced interval; and
an engaging portion deposited on an inner surface of the protruding edge and selectively connected to the engaging element of the lower flange.

11. The storage bottle as claimed in claim 4, wherein
the inner body has an engaging structure deposited around an external surface of the inner body adjacent to the opening;
the outer casing has
a top end;
a bottom end;
an upper flange axially and annularly formed on and protruding from the top end of the outer casing, and having an inner surface and an outer surface;
a lower flange axially and annularly formed on and protruding from the bottom end of the outer casing, deposited below the inner body, and having an outer surface;
an inner engaging segment deposited on the inner surface of the upper flange, and selectively engaging with the engaging structure of the inner body to hold the inner body in the outer casing;
an outer engaging segment deposited on the outer surface of the upper flange; and
an engaging element deposited on the outer surface of the lower flange;
the bottom base has
a protruding edge axially and annularly formed on and protruding front a top end of the bottom base, and connected to the lower flange of the outer casing to hold the bottom base below the inner body at a spaced interval; and
an engaging portion deposited on an inner surface of the protruding edge and selectively connected to the engaging element of the lower flange.

12. The storage bottle as claimed in claim 9, wherein
the inner body has an engaging structure deposited around an external surface of the inner body adjacent to the opening;
the outer casing has
a top end;
a bottom end;
an upper flange axially and annularly formed on and protruding from the top end of the outer casing, and having an inner surface and an outer surface;
a lower flange axially and annularly formed on and protruding from the bottom end of the outer casing, deposited below the inner body and having an outer surface;
an inner engaging segment deposited on the inner surface of the upper flange, and selectively engaging with the engaging structure of the inner body to hold the inner body in the outer casing;
an outer engaging segment deposited on the outer surface of the upper flange; and
an engaging element deposited on the outer surface of the lower flange;
the bottom base has
a protruding edge axially and annularly formed on and protruding from a top end of the bottom base, and connected to the lower flange of the outer casing to hold the bottom base below the inner body at a spaced interval; and
an engaging portion deposited on an inner surface of the protruding edge and selectively connected to the engaging element of the lower flange.
